(19) United States
(12) Patent Application Publication
(10) Pub. No.: US 2025/0246266 A1
(43) Pub. Date: Jul. 31, 2025

(54) GENOTYPING CYP2D6

(71) Applicant: GUARDANT HEALTH, INC., Palo Alto, CA (US)

(72) Inventor: Sante GNERRE, Mountain View, CA (US)

(21) Appl. No.: 18/897,596

(22) Filed: Sep. 26, 2024

Related U.S. Application Data
(60) Provisional application No. 63/586,835, filed on Sep. 29, 2023.

(51) Int. Cl. G16B 30/00; G16B 40/20

(52) U.S. Cl. CPC G16B 30/00; G16B 40/20

(57) ABSTRACT

Provided herein are methods and systems for allele typing and variant calling. Genotyping CYP2D6 is complicated by the fact that in some cases one or both of the alleles contain tandem rearrangements and/or copy number alterations. Furthermore, some alleles share 100% identical stretches with homologous regions, such as CYP2D7 and CYP2D8P.

### BACKGROUND

CYP2D6, a Phase I metabolizing enzyme, is notoriously difficult to accurately genotype. Multiple studies report discordant results between sequencing and single variant genotyping techniques. While small in size (˜4400 nucleotides from starting ATG to stop codon), the polymorphic nature of CYP2D6, as well as its surrounding locus add to the complexity of being able to comprehensively and correctly genotype it.

Accordingly, there is a need for methods for accurate allele typing of complex structures using sequence information.

### SUMMARY

Disclosed are methods of allele typing. Disclosed are methods of treatment based on allele typing.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, and determining, based on the numbers of sequence reads that aligned to each known allele sequence, for the one or more loci, the known allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence read families (i.e., number of nucleic acid molecules—a sequence read family may be a group of sequence reads corresponding to a single nucleic acid molecule) that aligned to each known allele sequence, and determining, based on the numbers of sequence read families that aligned to each known allele sequence, for the one or more loci, the known allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, generating, based on the numbers of sequence reads that aligned to each known allele sequence, one or more supersets of known allele sequences, and determining, based on a number of distinct reads in the one or more supersets of known allele sequences, for the one or more loci, the known allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence read families that aligned to each known allele sequence, generating, based on the numbers of sequence read families that aligned to each known allele sequence, one or more supersets of known allele sequences, and determining, based on a number of distinct read families in the one or more supersets of known allele sequences, for the one or more loci, the known allele sequences present at the one or more loci.

In some embodiments, the results of the systems and methods disclosed herein are used as an input to generate a report. The report may be in a paper or electronic format. For example, the determination of allele type (e.g., allele sequence), as determined by the methods and systems disclosed herein, can be displayed directly in such a report.

The various steps of the methods disclosed herein, or steps executed by the systems disclosed herein, may be carried out at the same or different times, in the same or different geographical locations, e.g., countries, and/or by the same or different people.

### BRIEF DESCRIPTION OF THE FIGURES

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings unless the context indicates otherwise. It will also be understood that some or all of the figures may be schematic representations for purposes of illustration and do not necessarily depict the actual relative sizes or locations of the elements shown.

FIG. 1A is a flow chart that schematically depicts exemplary method steps for allele typing.

FIG. 1B is a flow chart that schematically depicts another exemplary method steps for allele typing.

FIG. 2 shows an example of a system for allele typing.

FIG. 3 shows an example nucleic acid structures.

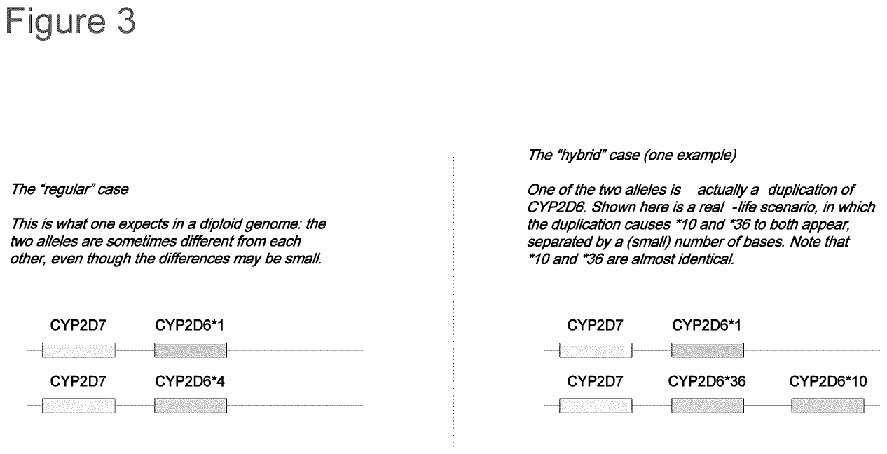

FIG. 4 shows an example rearrangement and other complex structures.

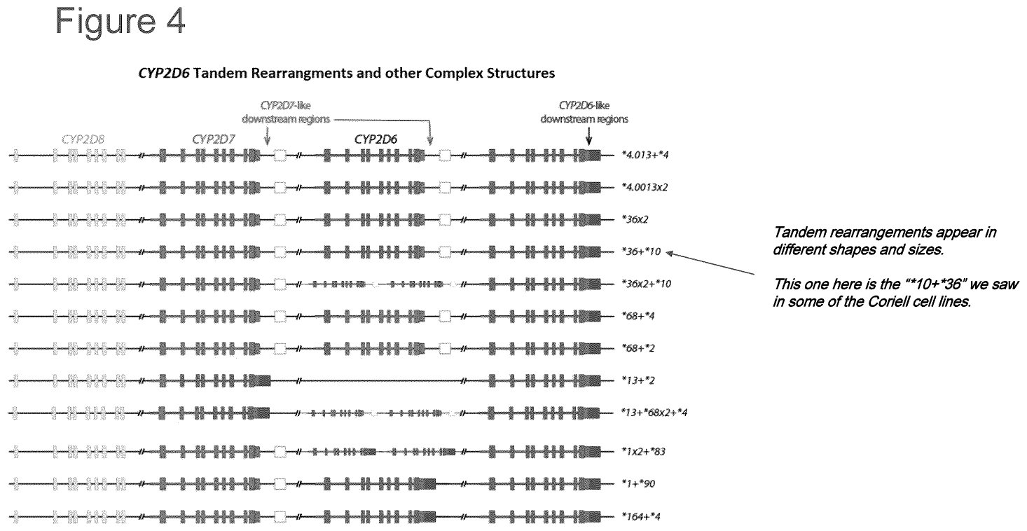

FIG. 5 shows an example sequence reads.

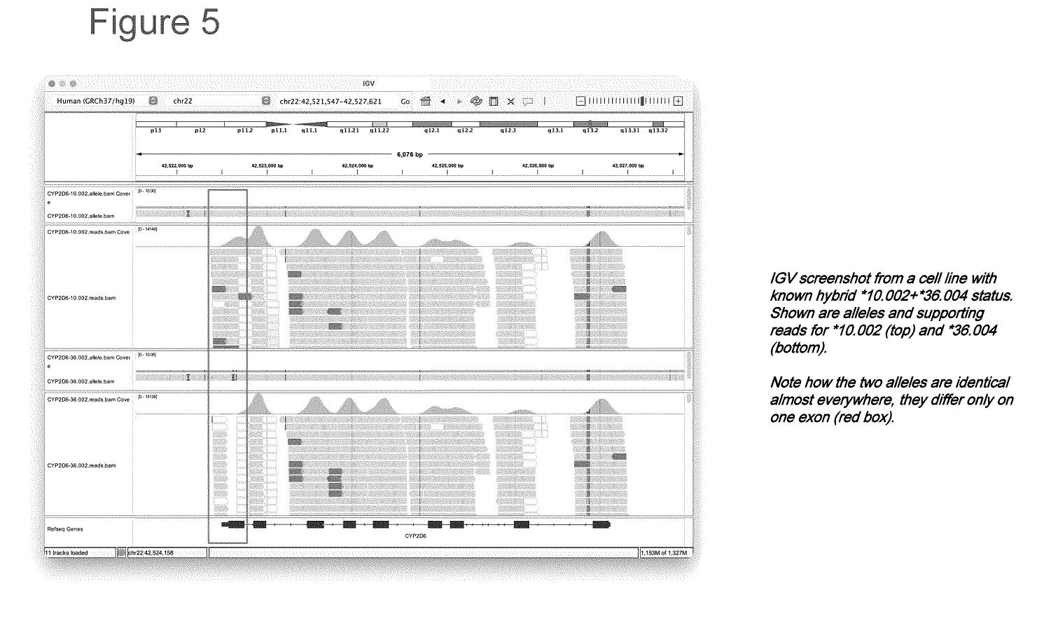

FIG. 6 shows an example graph data structure.

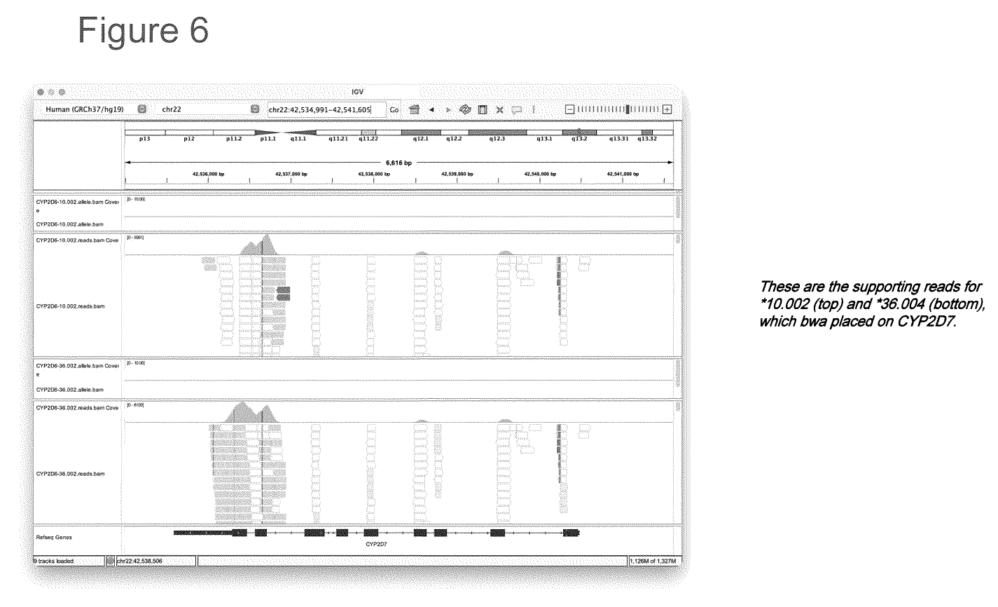

FIG. 7 shows an example different CYP2D6 alleles.

FIG. 8 shows an example comparison.

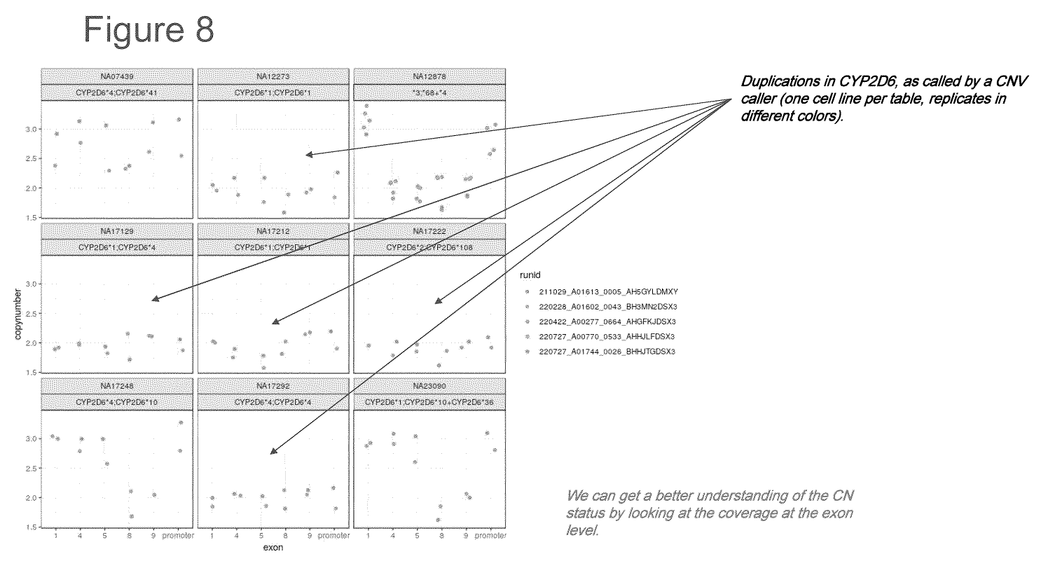

FIG. 9 shows an example comparison.

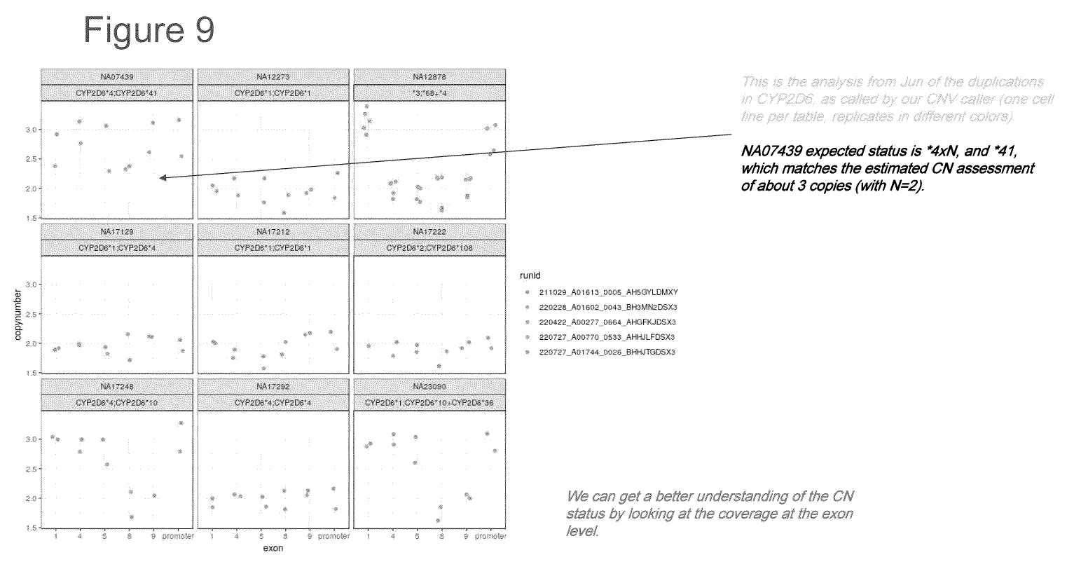

FIG. 10 shows an example comparison.

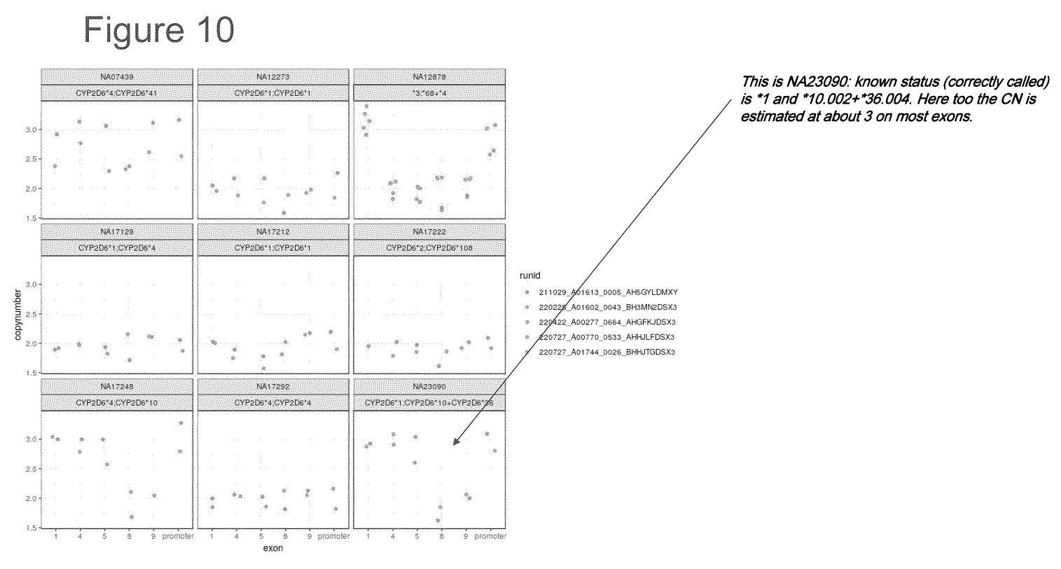

FIG. 11 shows an example comparison.

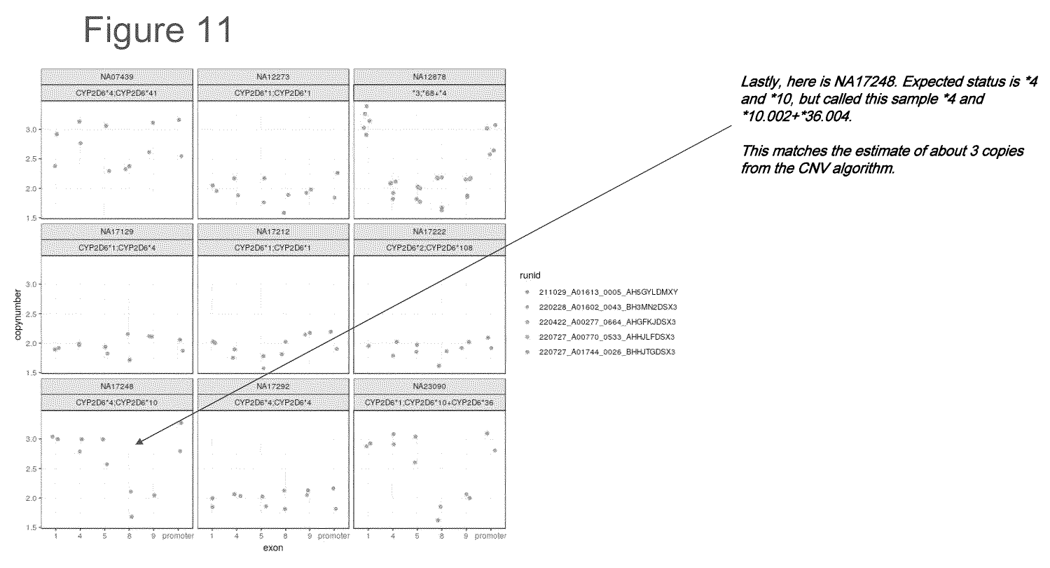

FIG. 12 shows example CNV calls.

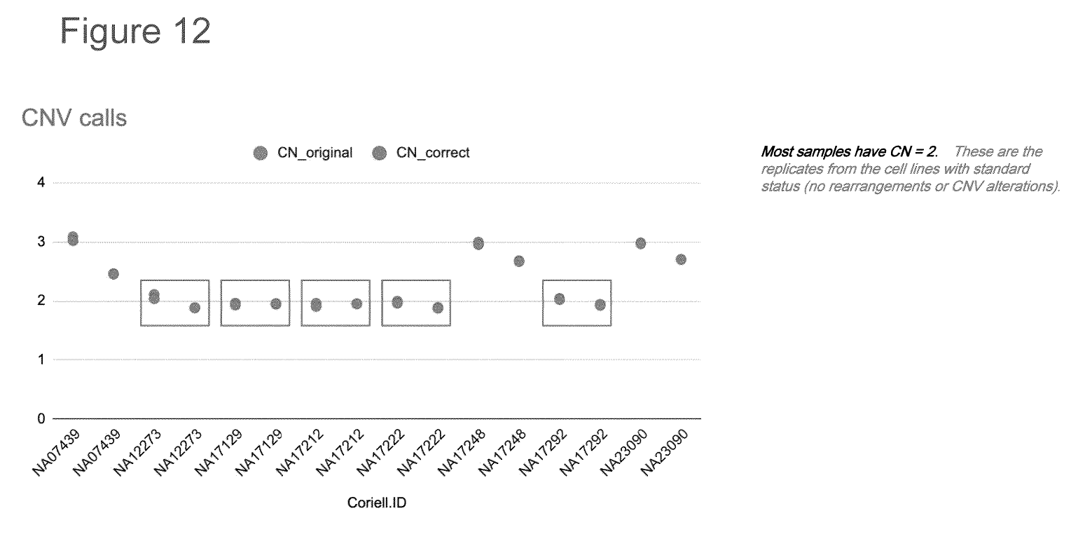

FIG. 13 shows example CNV calls.

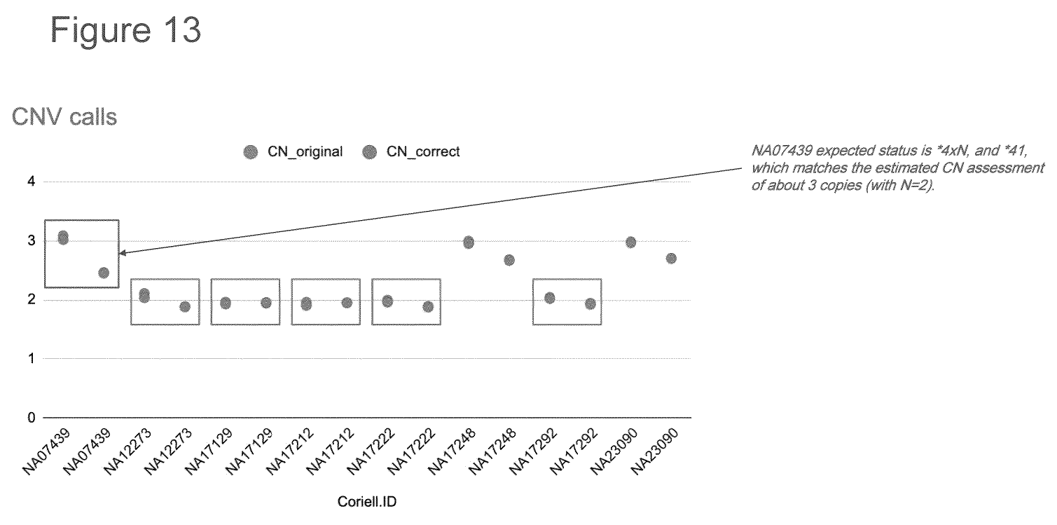

FIG. 14 shows example CNV calls.

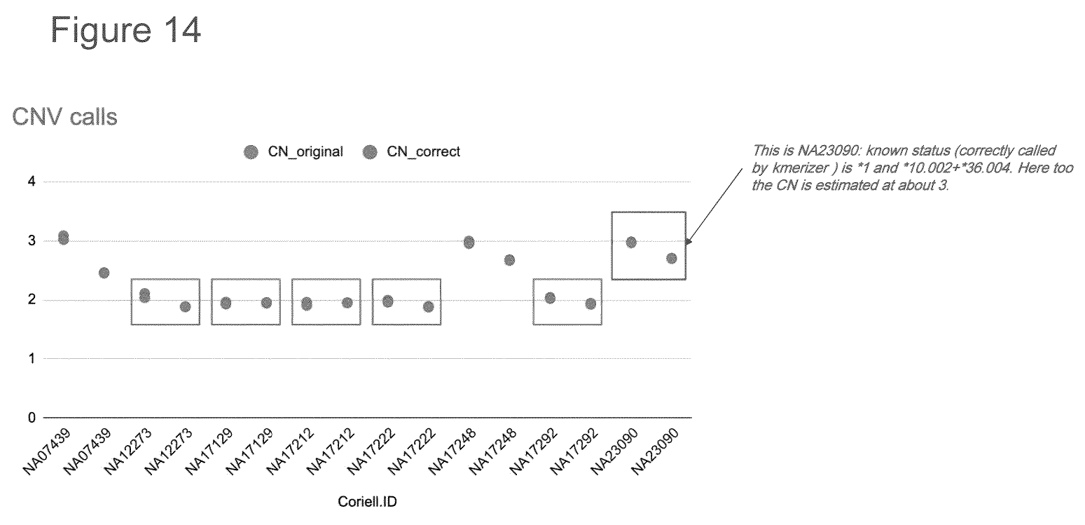

FIG. 15 shows example CNV calls.

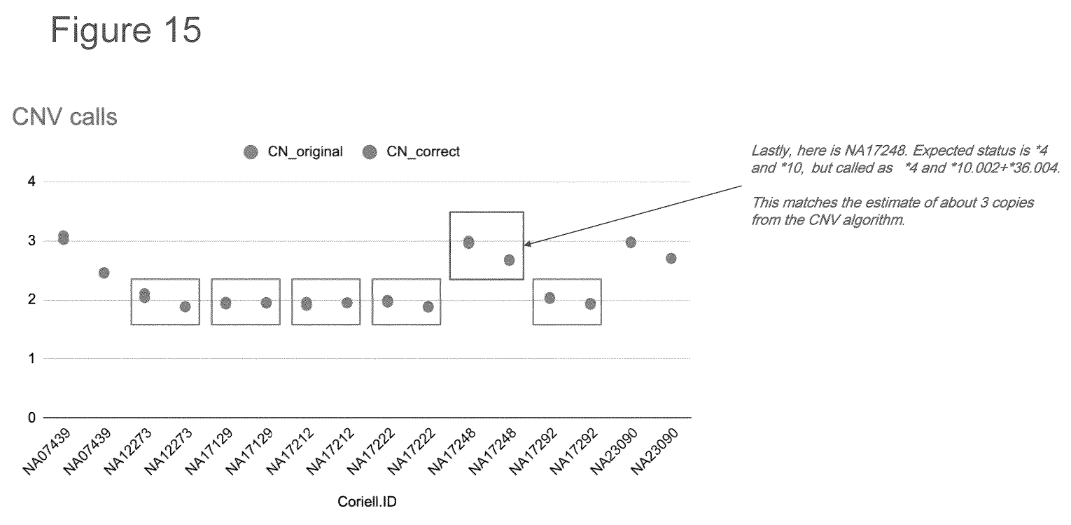

### I. DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise,” “comprises,” “comprising,” “contain,” “contains,” “containing,” “include,” “includes,” and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

### DETAILED DESCRIPTION

### II. INTRODUCTION

Provided herein are methods and systems for determining an allele type from a nucleic acid sample obtained from a test subject. In some aspects, the nucleic acid sample can be, but is not limited to, cell-free nucleic acid (cfNA), genomic DNA, or RNA. In an embodiment, the nucleic acid sample may be derived from a specific chromosome and/or from a specific region of a chromosome. In an embodiment, the nucleic acid sample may be derived from all or a portion of a metabolizing enzyme, such as CYP2D6.

### a. Metabolizing Enzyme CYP2D6

The first challenge to face is copy number variation (CNV), as CYP2D6 can be duplicated, multiplicated or deleted. Between zero all the way up to 12 copies of CYP2D6 on a single allele have been described to date. A simple assessment of the number of CYP2D6 genes present in a particular sample is seemingly straightforward; complications lie in the chimeras and/or hybrids that exist with its nearest neighbor and psuedogene, CYP2D7. CYP2D6 and CYP2D7 can form two main types of hybrids based on their structure-either CYP2D6/CYP2D7 or CYP2D7/CYP2D6 hybrids.

Even with correct calculation of the number of CYP2D6 genes and hybrids present in a particular sample, assessment of sequence variations is nearly impossible. CYP2D6 contains numerous sequence variations in CYP2D6, encompassing point mutations, insertions, deletions and the like. At issue is is deciding which CYP2D6 sequence variants should be interrogated. While commercially available CYP2D6 genotyping panels are purportedly available, an apparent drawback of genotyping panels designed to detect single sequence variants is the possibility of known and unknown mutations within the remaining, non-interrogated sequence of the gene. Here, even when sequence the entire gene using next generation sequencing techniques, along with software tools for analyzing and calling the CYP2D6 genotype, is the propensity for misalignment of sequence reads to the highly homologous CYP2D7 and nondetection of the structural variants (hybrids and/or duplications and multiplications).

### III. EXEMPLARY SYSTEMS AND METHODS

At step 104A, the data may be pre-processed. For example, step 104A may comprise constructing an allele k-mer data structure. The allele k-mer data structure may be a database. The allele k-mer data structure may be a flat file. The allele k-mer data structure may be any form of data structure. Constructing the allele k-mer data structure may comprise dividing the known allele sequences into a quantity of k-mers. For example, a quantity of k-mers having a length from about 100 nucleotides to about 200 nucleotides. In an embodiment, the quantity of k-mers may have a length of 143 nucleotides. Constructing the allele k-mer data structure may comprise associating each k-mer with metadata. The metadata may comprise, for example, an indication of a quantity of alleles that contain the k-mer and, for each allele that contains the k-mer, an allele identifier and a start position of the k-mer.

At step 106A, sequence processing may be performed. For example, step 106A may comprise obtaining (or otherwise determining, retrieving, receiving, etc.) sequence read pairs (e.g., test sequence reads) from a cell-free nucleic acid (cfDNA) sample obtained from a test subject. Step 106A may comprise performing an alignment between the test sequence reads and the known allele sequences. For example, step 106A may comprise performing an alignment between the test sequence reads and the k-mers in the allele k-mer data structure. The sequence processing may determine an allele(s) supported by a test sequence read(s). An allele may be supported by more than one test sequence read. A test sequence read may support more than one allele. In an embodiment, a test sequence read may be found to support an allele if the test sequence read aligns to the allele (e.g., a k-mer of the allele) with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the allele (e.g., a k-mer of the allele).

In an embodiment, step 106A may comprise determining a number of test sequence read families that support an allele(s) (e.g., a number of nucleic acid molecules that support an allele(s)). Each test sequence read may comprise a barcode. The barcode may identify the nucleic acid molecule (e.g., test sequence read family) with which the test sequence read is associated. In an embodiment, a test sequence read family may be found to support an allele if the test sequence read family aligns to the allele with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read family and the allele (e.g., a k-mer of the allele).

At step 108A, a clustering operation may be performed. The alleles may be sorted by the number of supporting test sequence reads (or by the number of supporting test sequence read families) and one or more allele supersets may be constructed. An allele superset may be constructed by determining a first allele associated with a highest number of supporting test sequence reads (or associated with a highest number of supporting test sequence read families). The first allele may form the basis of an allele superset. Additional alleles may be added to the allele superset if a given allele is associated with supporting test sequence reads (or supporting test sequence read families) are themselves a subset of the supporting test sequence reads (or supporting test sequence read families) of the first allele. Alleles that are not incorporated into the allele superset of the first allele may be used to construct one or more additional allele supersets in a similar fashion.

An allele superset may be a data structure. An allele superset may be a database. An allele superset may be a flat file. An allele superset may comprise a representation of a Hasse diagram. A Hasse diagram is a representation of the relation of elements of a partially ordered set with an implied upward orientation. A point, or node, may represent each element of the partially ordered set and nodes may be joined with a line segment according to the following rules: 1) if p<q in the partially ordered set, then the point corresponding to p appears lower in the drawing than the point corresponding to q; 2) the two points p and q will be joined by a line segment if p is related to q. In an embodiment, the Hasse diagram may be represented as a graph data structure, such as a directed acyclic graph (DAG) and/or the like. For example, a DAG comprising a line from node A to node B if node A strictly contains node B and there is no node C such that node A strictly contains node C and node C strictly contains node B.

At step 110A, an allele may be classified. For example, at step 110A an allele type may be determined for a given allele. The allele may be classified based on the one or more allele supersets. In an embodiment, in the event only one allele superset is constructed the first allele of the superset may be classified as the allele present at the locus (e.g., haploid locus) of the chromosome. In an embodiment, in the event that a plurality of allele supersets are constructed, the first alleles of the two supersets having a cumulative largest number of distinct supporting test sequence reads (or a cumulative largest number of distinct supporting test sequence read families) may be classified as the alleles present at the locus (e.g., diploid locus) of the chromosome.

The classification of the allele(s) may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering immunotherapy, administering chemotherapy, administering radiation therapy, or performing surgery to resect all or a portion of the tumor. The methods may comprise assisting in a communication of determination of the classification of the allele(s) to a subject associated with the test sample.

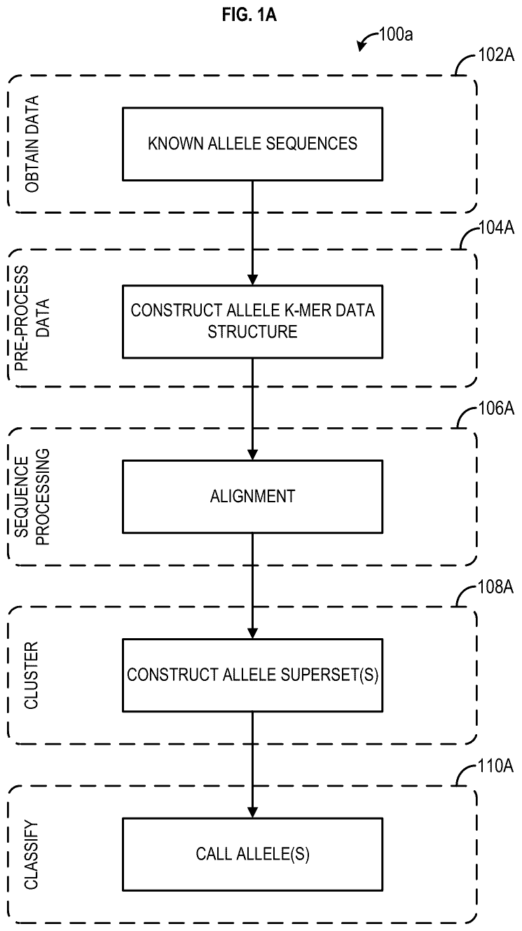

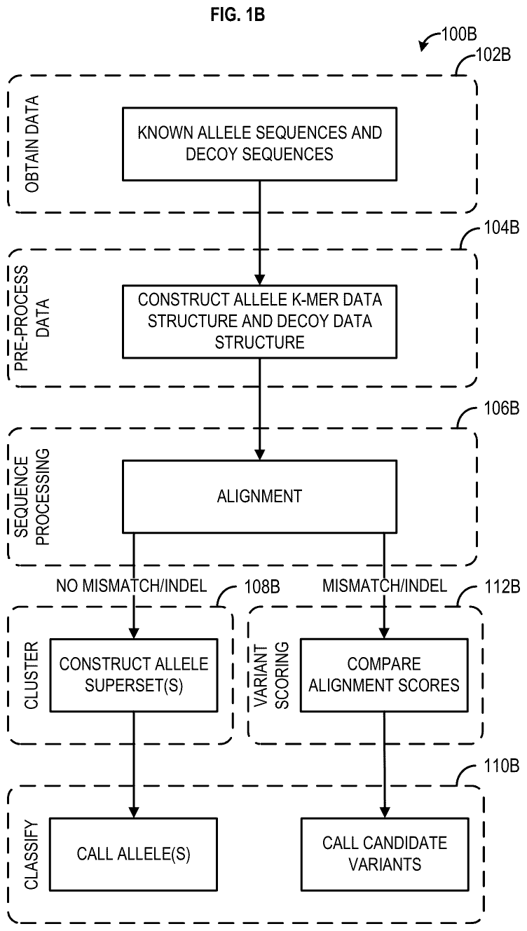

FIG. 1B is a flow chart that schematically depicts an example technique for allele typing and/or variant calling in a cell-free nucleic acid (cfDNA) sample obtained from a test subject. Allele typing may be used to determine one or more alleles present at a locus of a chromosome. Variant calling may be used to identify the presence of a known, or unknown variant. Variant calling may be used to characterize cancer progression. As shown, a method 100B, at step 102B, may comprise obtaining data. The data may comprise sequence data, such as allele sequence data and/or decoy sequence data.

The decoy sequences are sequences of genomic material (human, in general) similar to the sequences we want to look at (for example, the regions we want to genotype). These are not already part of the reference because they encode an alternate form of a region or gene (hence the name “alt”). The problem for us is that we deploy targeted sequencing, which is a way to select only molecules from portions of genome matching some specified region (these “specified regions” are called probes, or baits, and in our case are 120 bases long): what happens is that sometimes a probe designed to capture molecules from a region of interest, instead captures molecules from one of these “alt” sequences. We can detect this because in these cases the read (or read pair) aligns better on the decoy than on the human reference.

In an embodiment, the decoy sequences may comprise decoy sequences selected to identify contamination in the test sample. The one or more decoy sequences may comprise one or more non-human reference sequences. For example, the one or more decoy sequences may comprise bovine reference sequences, rat reference sequences, microbial reference sequences, combinations thereof and the like. Any test sequences pairs aligning to a non-human decoy sequence may be used to support a conclusion that the test sample has been contaminated with DNA from sources other than the test subject. The idea is the same as above, only we use here as “decoy” the sequence of our suspected contaminants. For example, assume we suspect there is some cow DNA in our sample (which happened!), then we add the whole cow genome to our decoy list, and when we align a read pair to both the human reference and to the cow genome (the decoy), we find that the contaminant molecules align better on cow than on human.

At step 104B, the data may be pre-processed. For example, step 104B may comprise constructing an allele k-mer data structure. The allele k-mer data structure may be a database. The allele k-mer data structure may be a flat file. The allele k-mer data structure may be any form of data structure. Constructing the allele k-mer data structure may comprise dividing the known allele sequences into a quantity of k-mers. For example, a quantity of k-mers having a length from about 100 nucleotides to about 200 nucleotides. In an embodiment, the quantity of k-mers may have a length of 143 nucleotides. Constructing the allele k-mer data structure may comprise associating each k-mer with metadata. The metadata may comprise, for example, an indication of a quantity of alleles that contain the k-mer and, for each allele that contains the k-mer, an allele identifier and a start position of the k-mer.

For example, step 104B may comprise constructing a decoy data structure. The decoy data structure may be a database. The decoy data structure may be a flat file. The decoy data structure may be any form of data structure. Structuring the algorithm, like this (ie, with a target sequence plus decoy sequence) allows us to keep some flexibility. The idea is that we can always add to the decoy any number of as-yet unknown “problematic” sequence, where in this case problematic means sequence similar to the one of our targets (in other words, sequence we could accidentally pick-up with our targeted sequencing tech dev, instead of the target region).

At step 106B, sequence processing may be performed. For example, step 106B may comprise obtaining (or otherwise determining, retrieving, receiving, etc.) sequence reads (e.g., test sequence reads) from a cell-free nucleic acid (cfDNA) sample obtained from a test subject. Step 106B may comprise performing an alignment between the test sequence reads and the known allele sequences. For example, step 106B may comprise performing an alignment between the test sequence reads and the k-mers in the allele k-mer data structure. The sequence processing may determine an allele(s) supported by a test sequence read(s). An allele may be supported by more than one test sequence read. A test sequence read may support more than one allele. In an embodiment, a test sequence read may be found to support an allele if the test sequence read aligns to the allele (e.g., a k-mer of the allele) with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the allele (e.g., a k-mer of the allele) indicating no mismatches and no indels. In an embodiment, the threshold percent identity may be less than 100%, requiring an “imperfect” match between a test sequence read and the allele (e.g., a k-mer of the allele) indicating at least one mismatch and/or at least one indel. An indication of percent identity may be determined for each alignment and stored for later processing. The results of an alignment may be represented by an alignment score, described in further detail with regard to the alignment component 215. The alignment score may equal the sum of the number of mismatches and the number of indels.

In an embodiment, step 106B may comprise determining a number of test sequence read families that support an allele(s) (e.g., a number of nucleic acid molecules that support an allele(s)). Each test sequence read may comprise a barcode. The barcode may identify the nucleic acid molecule (e.g., test sequence read family) with which the test sequence read is associated. In an embodiment, a test sequence read family may be found to support an allele if the test sequence read family aligns to the allele with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read family and the allele (e.g., a k-mer of the allele).

Step 106B may comprise performing an alignment between the test sequence reads and the decoy sequences. For example, step 106B may comprise performing an alignment between the test sequence reads and the decoy sequences in the decoy data structure. The sequence processing may determine a decoy sequence(s) supported by a test sequence read(s). In an embodiment, a test sequence read may be found to support a decoy sequence if the test sequence read aligns to the decoy sequence with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the decoy sequence) indicating no mismatches and no indels. An indication of percent identity may be determined for each alignment and stored for later processing. In an embodiment, one or more test sequence reads that align to one or more decoy sequences with 100% identity may be discarded and not used for further processing. In an embodiment, to the extent the decoy sequences comprise one or more non-human sequences, any test sequence reads that match to a non-human decoy sequence with 100% identify may be used to support identification of the test sample as being contaminated. A notification associated with potential contamination may be generated and/or sent.

The results of an alignment may be represented by an alignment score, described in further detail with regard to the alignment component 215. The alignment score may equal the sum of the number of mismatches and the number of indels.

At step 108B, a clustering operation may be performed based on alignments between the test sequence reads and the known allele sequences. The known alleles may be sorted by the number of supporting test sequence reads (or by the number of supporting test sequence read families) and one or more allele supersets may be constructed. An allele superset may be constructed by determining a first allele associated with a highest number of supporting test sequence reads (or associated with a highest number of supporting test sequence read families). The first allele may form the basis of an allele superset. Additional alleles may be added to the allele superset if a given allele is associated with supporting test sequence reads (or supporting test sequence read families) are themselves a subset of the supporting test sequence reads (or supporting test sequence read families) of the first allele. Alleles that are not incorporated into the allele superset of the first allele may be used to construct one or more additional allele supersets in a similar fashion.

An allele superset may be a data structure. An allele superset may be a database. An allele superset may be a flat file. An allele superset may comprise a representation of a Hasse diagram. A Hasse diagram is a representation of the relation of elements of a partially ordered set with an implied upward orientation. A point, or node, may represent each element of the partially ordered set and nodes may be joined with a line segment according to the following rules: 1) if p<q in the partially ordered set, then the point corresponding to p appears lower in the drawing than the point corresponding to q; 2) the two points p and q will be joined by a line segment if p is related to q. In an embodiment, the Hasse diagram may be represented as a graph data structure, such as a directed acyclic graph (DAG) and/or the like. For example, a DAG comprising a line from node A to node B if node A strictly contains node B and there is no node C such that node A strictly contains node C and node C strictly contains node B.

At step 110B, an allele may be classified. For example, at step 110B an allele type may be determined for a given allele. The allele may be classified based on the one or more allele supersets. In an embodiment, in the event only one allele superset is constructed the first allele of the superset may be classified as the allele present at the locus (e.g., haploid locus) of the chromosome. In an embodiment, in the event that a plurality of allele supersets are constructed, the first alleles of the two supersets having a cumulative largest number of distinct supporting test sequence reads (or a cumulative largest number of distinct supporting test sequence read families) may be classified as the alleles present at the locus (e.g., diploid locus) of the chromosome.

The classification of the allele(s) may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering immunotherapy, administering chemotherapy, administering radiation therapy, or performing surgery to resect all or a portion of the tumor. The methods may comprise assisting in a communication of determination of the classification of the allele(s) to a subject associated with the test sample.

At step 110B, the test sequence read pairs associated with a germline alignment score that is greater than a decoy alignment score may be analyzed to determine and/or identify the test sequence read pairs as a variant. Variant calling is the process of identifying true differences between sequence reads of test samples and a reference sequence. Variant calling may be performed as further described with regard to the variant caller component 219 below. In an embodiment, the test sequence read pairs may be identified as a somatic variant. In an embodiment, the test sequence read pairs may be identified as a variant that is a candidate variant associated with a somatic event. At step 110B, candidate variants may be identified in the test sequence read pairs. In one embodiment, the candidate variants may be identified by comparing the test sequence read pairs to a reference sequence of a target region of a reference genome (e.g., human reference genome hg19). Edges of the test sequence read pairs may be aligned to the reference sequence and the genomic positions of mismatched edges and mismatched nucleotide bases adjacent to the edges recorded as the locations of candidate variants. In some embodiments, the genomic positions of mismatched nucleotide bases to the left and right edges are recorded as the locations of called variants. Additionally, candidate variants may be identified based on the sequencing depth of a target region. In particular, more confidence may be obtained in identifying variants in target regions that have greater sequencing depth, for example, because a greater number of sequence reads help to resolve (e.g., using redundancies) mismatches or other base pair variations between sequences.

In an embodiment, the reference sequence used for variant calling may comprise one or more reference sequences. The one or more reference sequences may be selected to identify contamination in the test sample. The one or more reference sequences may comprise one or more non-human reference sequences. For example, the one or more reference sequences may comprise a bovine reference sequences, rat reference sequences, microbial reference sequences, combinations thereof, and the like. Any test sequences pairs identified as a non-human variant may be used to support a conclusion that the test sample has been contaminated with DNA from sources other than the test subject.

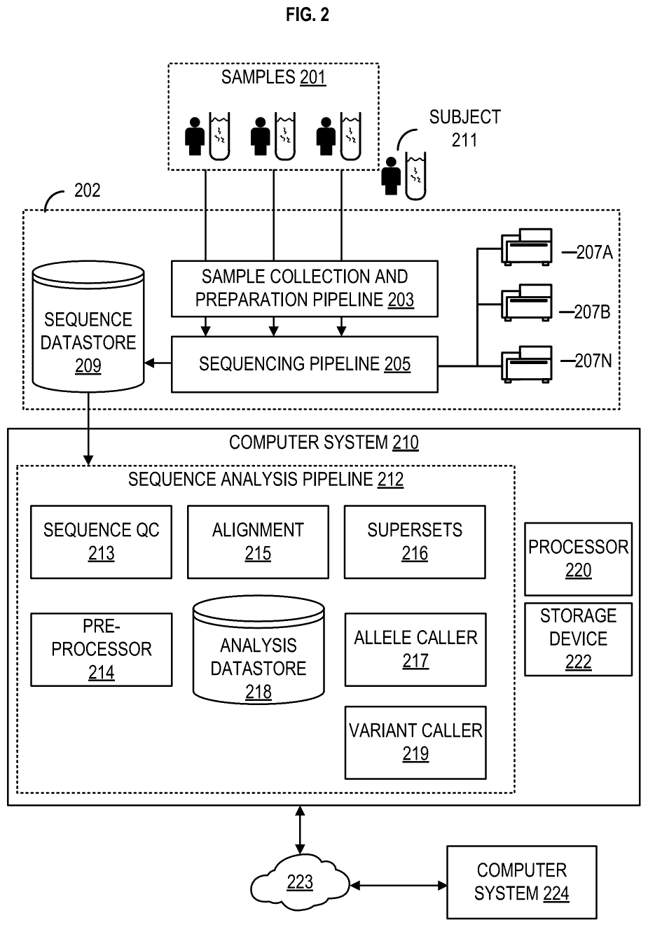

FIG. 2 illustrates an example of a system 200 for determining an allele type and/or a variant of a test subject 211, according to an embodiment of the present disclosure. The system 200 may process one or more samples 201 from the subject 211 to generate sequence reads. The system 200 may include a laboratory system 202, a computer system 210, and/or other components. It should be noted that the laboratory system 202 and the computer system 210 may be remote from one another, and connected to one another through a computer network (not illustrated). The laboratory system 202 may include a sample collection and preparation pipeline 203, a sequencing pipeline 205, a sequence read datastore 209, and/or other components. The sequencing pipeline 205 may include one or more sequencing devices 207 (illustrated in FIG. 2 as sequencing devices 207a . . . n).

The methods of this disclosure may have a wide variety of uses in the manipulation, preparation, identification, quantification, and/or analysis of cell-free nucleic acids. As shown in FIG. 2, the sample collection and preparation pipeline 203 may include obtaining cfDNA reference samples 201 from one or more reference subjects and a cfDNA test sample 211 from a test subject. As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g., healthy) bodily cells.

### a. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.).

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

### b. Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, U.S. patent applications 20010053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to create a unique sequence that may be individually tracked. In an embodiment, techniques for discriminating true genomic alterations from technical errors may be used as described in Lee, et al., “Accurate Detection of Rare Mutant Alleles by Target Base-Specific Cleavage with the CRISPR/Cas9 System,” ACS Synth. Biol. 2021, 10, 6, 1451-1464 May 19, 2021, incorporated herein by reference in its entirety. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1,first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

### c. Nucleic Acid Amplification

Sample nucleic acids flanked by adapters are typically amplified by PCR and other amplification methods using nucleic acid primers binding to primer binding sites in adapters flanking a DNA molecule to be amplified as part of the sample collection and preparation pipeline 203. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

In some aspects, amplification can occur pre and/or post enrichment.

### d. Nucleic Acid Enrichment

In some embodiments, sequences are enriched prior to sequencing the nucleic acids as part of the sample collection and preparation pipeline 203. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. In some embodiments, targeted regions of interest may be enriched using CRISPR mediated enrichment. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

In some embodiments, a probe can be designed to be specific to the alleles of interest. Thus, different alleles from the same gene have an equal chance to be captured. In some aspects, after enrichment, amplification (as described above) can be performed.

### e. Nucleic Acid Sequencing

As shown in FIG. 2, after extraction and isolation of cfDNA from samples via the sample collection and preparation pipeline 203, the cfDNA may be sequenced via the sequencing pipeline 205 including one or more sequencing devices 207. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain alleles of interest. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Additional details regarding nucleic acid sequencing, including the formats and applications described herein are also provided in, for example, Levy et al., Annual Review of Genomics and Human Genetics, 17:95-115 (2016), Liu et al., J. of Biomedicine and Biotechnology, Volume 2012, Article ID 251364:1-11 (2012), Voelkerding et al., Clinical Chem., 55:641-658 (2009), MacLean et al., Nature Rev. Microbiol., 7:287-296 (2009), Astier et al., J Am Chem Soc., 128 (5): 1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### f. Sequencing Panel

To improve the likelihood of detecting genomic regions of interest the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced).

Genes included in the panel for sequencing can include the fully transcribed region, the promoter region, enhancer regions, regulatory elements, and/or downstream sequence. In some embodiments, only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

At least one full exon from each different gene in a panel of genes may be sequenced. In some aspects, all of the exons of a gene may be sequenced. The sequenced panel may comprise all or some exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. In some aspects, a selected panel may comprise all of the exons of a gene. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be 50 kb to 10 Mb in size. The sequencing panel can be 500 kb to 5 Mb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, 150 kb, 200 kb, 250 kb, 300 kb, 350 kb, 400 kb, 450 kb, or 500 kb in size. The sequencing panel may be less than 100 kb, 90 kb, 80 kb, 70 kb, 60 kb, or 50 kb in size. The sequencing panel can be at least 1 Mb, 2 Mb, 3 Mb, 4 Mb, 5 Mb, 6 Mb, 7 Mb, 8 Mb, 9 Mb, or 10 Mb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, utilizing the sequencing pipeline 205, the panel may be subjected to one or more of: whole-genome bisulfite sequencing (WGBS) interrogating genome-wide methylation patterns, whole-genome sequencing (WGS), and/or targeted sequencing approaches interrogating copy-number variants (CNVs) and single-nucleotide variants (SNVs).

### g. Sequence Analysis Pipeline

In an embodiment, after sequencing, sequence reads and any associated data may be stored in the sequence datastore 209. The sequence reads can be stored in any format. The sequence datastore 209 may be local and/or remote to a location where sequencing is performed. As shown in FIG. 2, the stored reads may be subjected to a sequence analysis pipeline 212.

i. Sequence Quality Control

The sequence analysis pipeline 212 may include a sequence quality control (QC) component 213 that may filter sequence reads from the laboratory system 102. The sequence QC component 213 may assign a quality score to one or more sequence reads. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 60%, 70%, 80%, 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set.

Sequence reads that meet a specified quality score threshold may be mapped to a reference genome by the sequence QC component 213. After mapping alignment, sequence reads may be assigned a mapping score. A mapping score may be a representation of sequence reads mapped back to the reference sequence indicating whether each position is or is not uniquely mappable. Sequence reads with a mapping score at least 60%, 70%, 80%, 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

ii. Pre-Processor

A pre-processor 214 may retrieve/receive data from the analysis datastore 218. For example, the pre-processor 214 may retrieve/receive data representing the plurality of known allele sequences, the plurality of test sequence reads, and/or the plurality of decoy sequences. The pre-processor 214 may also be configured to retrieve sequence data from another source (e.g., an external source).

The pre-processor 214 may be configured to divide the known allele sequences into a plurality of k-mer sequences. In other example, k may be from about 25 to about 250. For example, k may be 135 or 140. In an embodiment, k may be 125-175 nucleotides, 130-160 nucleotides, 135-155 nucleotides, 140-150 nucleotides in length. In an embodiment, the k may be 140, 141, 142, 143, 144, or 145 nucleotides in length.

The pre-processor 214 may create a database comprising the k-mer sequences and additional data. The pre-processor 214 may create a data structure comprising the k-mer sequences and additional data. The data structure may be, for example, a table or a flat file.

iii. Alignment Component

An alignment component 215 may retrieve/receive data from the analysis datastore 218. For example, the alignment component 215 may retrieve/receive data representing the plurality of known allele sequences, k-mer sequences generated from the plurality of known allele sequences, the plurality of test sequence reads, and/or the plurality of decoy sequences.

In various embodiments, the alignment component 215 may be configured to align a test sequence read to a reference sequence or another test sequence read. The alignment component 215 may be configured to align a test sequence read to one or more k-mer sequences generated from the plurality of known allele sequences. The alignment component 215 may be configured to align a test sequence read (e.g., pair) to one or more decoy sequences.

An alignment score is a score indicating a similarity of two sequences determined using an alignment method. In some implementations, an alignment score accounts for number of edits (e.g., deletions, insertions, and substitutions of characters in the string). In some implementations, an alignment score accounts for a number of matches. In some implementations, an alignment score accounts for both the number of matches and a number of edits. In some implementations, the number of matches and edits are equally weighted for the alignment score. For example, an alignment score can be calculated as: #of matches- #of insertions- #of deletions- #of substitutions. In other implementations, the numbers of matches and edits can be weighted differently. For example, an alignment score can be calculated as: #of matches×5- #of insertions×4- #of deletions×4- #of substitutions×6.

Pairwise alignment generally involves placing one sequence along part of target, introducing gaps according to an algorithm, scoring how well the two sequences match, and preferably repeating for various positions along the reference. The best-scoring match is deemed to be the alignment and represents an inference of homology between alignment portions of the sequences. In some embodiments, scoring an alignment of a pair of nucleic acid sequences involves setting values for the scores of substitutions and indels. When individual bases are aligned, a match or mismatch contributes to the alignment score by a substitution probability, which could be, for example, 1 for a match and −0.33 for a mismatch. An indel deducts from an alignment score by a gap penalty, which could be, for example, −1. Gap penalties and substitution probabilities can be based on empirical knowledge or a priori assumptions about how sequences evolve. Their values affect the resulting alignment. Particularly, the relationship between the gap penalties and substitution probabilities influences whether substitutions or indels will be favored in the resulting alignment.

By way of example, the alignment component 215 may utilize a Burrows-Wheeler Aligner (BWA). Generally, the length of the test sequence read can be substantially less than the length of the k-mer sequences generated from the plurality of known allele sequences. The test sequence read and the k-mer sequences can include a sequence of symbols. The alignment of the test sequence read and the k-mer sequences can include a limited number of mismatches between the symbols of the test sequence read and the symbols of the k-mer sequences. Generally, the test sequence read can be aligned to a portion of the k-mer sequences in order to minimize the number of mismatches between the test sequence read and the k-mer sequences.

In particular embodiments, the symbols of the test sequence read and the k-mer sequence can represent the composition of biomolecules. For example, the symbols can correspond to identity of nucleotides in a nucleic acid, such as RNA or DNA. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

In an embodiment, the alignment component 215 may be configured to determine those test sequence reads that have an identical, or substantially identical, alignment to one or more k-mer sequences.

Two nucleic acid sequences or polypeptide sequences are said to be “identical” if the sequence of nucleotides or amino acid residues, respectively, in the two sequences is the same when aligned for maximum correspondence as described herein. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. The term “substantially identical,” used in the context of two nucleic acids or polypeptides, refers to a sequence that has at least 50% sequence identity with a reference sequence. Percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein, e.g., BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.).

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

Nucleic acid or protein sequences that are substantially identical to a reference sequence include “conservatively modified variants.” With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

Once the alignment component 215 has aligned the plurality of test sequence reads to one or more k-mers generated from the known allele sequences, a list of test sequence reads that aligned to (supported) a k-mer sequence of that allele can be generated for each allele. In an embodiment, only test sequence reads that align identically (e.g., no mismatches and no indels) to a k-mer sequence are included in the list. In an embodiment, only test sequence reads that align substantially identically (e.g., at least one mismatch and/or at least one indel) to a k-mer sequence are included in the list. In an embodiment, the alignment component can discard the actual alignment. In an embodiment, a test sequence read may align (identically or substantially identically) to a plurality of alleles. Each test sequence read may be associated with a test sequence read identifier. Accordingly, for each allele, a list of test sequence read identifiers associated with the supporting test sequence reads may be generated. A list of test sequence reads that aligned to a decoy sequence may also be generated. In an embodiment, only test sequence reads that align identically (e.g., no mismatches and no indels) to a decoy sequence are included in the list. In an embodiment, only test sequence reads that align substantially identically (e.g., at least one mismatch and/or at least one indel) to a decoy sequence are included in the list. The alignment component 215 may be configured to discard any test sequence reads that aligned to a decoy sequence with no mismatches and no indels.

iv. Superset Component

A cluster component 216 may retrieve/receive data from the analysis datastore 218. For example, the cluster component 216 may retrieve/receive data representing the plurality of known allele sequences, k-mer sequences generated from the plurality of known allele sequences, the plurality of test sequence reads, and results from the alignment component 215.

In an embodiment, a superset of one or more of the plurality of known allele sequences may be computationally generated by constructing one or more graph data structures. The graph data structure may comprise nodes (also referred to as vertices) representing known allele sequences and edges connecting the nodes indicating that supporting reads of one node are a subset of the supporting reads of the other node. Graph data structure construction may be parallelized given the computationally intensive nature of such construction.

In an embodiment, the graph data structure is stored in a memory subsystem (e.g., FIG. 2, memory 222), which may include pointers to identify a physical location in the memory 222 where each vertex is stored. Typically, the nodes in a graph data structure each represent an element in a set, while the edges represent relationships among the elements. The graph data structure may comprise a directed graph, a tree, a directed acyclic graph (DAG), and/or the like. A directed graph is one in which the edges have a direction. A tree is a type of directed graph data structure having a root node, and a number of additional nodes that are each either an internal node or a leaf node. The root node and internal nodes each have one or more “child” nodes and each is referred to as the “parent” of its child nodes. Leaf nodes do not have any child nodes. Edges in a tree are conventionally directed from parent to child. In a tree, nodes have exactly one parent. A generalization of trees, known as a directed acyclic graph (DAG), allows a node to have multiple parents, but does not allow the edges to form a cycle.

In an embodiment, the graph data structure (superset) may represent a Hasse diagram. For a given locus of the chromosome, the alleles may be sorted by the number of supporting test sequence reads. A graph data structure may be constructed by determining a first allele associated with a highest number of supporting test sequence reads. The first allele may form the basis of the graph data structure (e.g., top level node). The supporting test sequence reads of the first allele may define a set of supporting test sequence reads. Additional alleles may be added to the graph data structure if a given allele is associated with supporting test sequence reads are themselves a subset of the set of supporting test sequence reads of the first allele. Alleles that are not incorporated into the allele superset of the first allele may be used to construct one or more additional allele supersets in a similar fashion. By way of example, a given allele may have the highest number of supporting test sequence reads and each supporting test sequence read may be associated with a test sequence read identifier. In this instance, a set may be formed of the test sequence read identifiers of the supporting test sequence reads for the first allele. In a basic example, the first allele may be supported by test sequence reads having identifiers “1,” “2,” “3,” and “4.” The set of test supporting reads may be expressed as A={1, 2, 3, 4}. The power set of A, P(A), is the set of all subsets of A. For A={1, 2, 3, 4}: P(A)={Ø, {1}, {2}, {3}, {4}, {1, 2}, {1, 3}, {1, 4}, {2, 3}, {2, 4}, {3, 4}, {1, 2, 3}, {1, 2, 4}, {1, 3, 4}, {2, 3, 4}, {1, 2, 3, 4}}.

In an embodiment, the graph data structure (e.g., representing a superset) is stored in a memory subsystem (e.g., FIG. 2, memory 222) using adjacency techniques, which may include pointers to identify a physical location in the memory 222 where each vertex is stored. In an embodiment, the graph data structure is stored in the memory 222 using adjacency lists. In some embodiments, there is an adjacency list for each vertex.

In some embodiments, fast random access is supported and graph object storage are implemented with index-free adjacency in that every element contains a direct pointer to its adjacent elements, which obviates the need for index look-ups, allowing traversals to be very rapid. Index-free adjacency is another example of low-level, or hardware-level, memory referencing for data retrieval. Specifically, index-free adjacency can be implemented such that the pointers contained within elements are references to a physical location in memory.

Since a technological implementation that uses physical memory addressing such as native pointers can access and use data in such a lightweight fashion without the requirement of separate index tables or other intervening lookup steps, the capabilities of a given computer, e.g., any modern consumer-grade desktop computer, are extended to allow for full operation of a genomic-scale graph (e.g., the graph data structure 700 that represents a superset of known allele sequences). Thus storing graph elements (e.g., nodes and edges) using a library of objects with native pointers or other implementation that provides index-free adjacency actually improves the ability of the technology to provide storage, retrieval, and alignment for sequence information since it uses the physical memory of a computer in a particular way.

v. Allele Caller—Selection of Alleles From Supersets

An allele caller 217 may retrieve/receive data from the analysis datastore 218. For example, the allele caller 217 may retrieve/receive data representing the plurality of known allele sequences, k-mer sequences generated from the plurality of known allele sequences, the plurality of test sequence reads, results from the alignment component 215, and/or one or more graph data structures (supersets) generated by the cluster component 216.

The allele caller 217 may be configured to determine an allele type for a given allele. The allele caller 217 may be configured to classify an allele based on the one or more graph data structures (supersets).

In an embodiment, in the event only one graph data structure (superset) is constructed (e.g., all known allele sequences fall within a single graph data structure) the allele (the first allele) associated with the root node of the graph data structure may be classified as the allele present at the locus (e.g., haploid locus) of the chromosome.

In an embodiment, in the event that a plurality of graph data structures (supersets) are constructed, the alleles (the first alleles) associated with the root nodes of the two supersets having a cumulative largest number of distinct supporting test sequence reads may be classified as the alleles present at the locus (e.g., diploid locus) of the chromosome.

A set operation may be performed on combinations of root nodes to determine the two root nodes having a cumulative largest number of distinct supporting test sequence reads. In an embodiment a union operation (U) may be used.

vi. Variant Caller

A variant caller 219 may retrieve/receive data from the analysis datastore 218. For example, the variant caller 219 may retrieve/receive data representing a plurality of sequence reads. The variant caller 219 may retrieve test sequence reads that aligned to a decoy sequence and to a known allele with at least one mismatch and/or at least one indel and that had a greater alignment score to the known allele. The test sequence reads may be analyzed to determine one or more variants. Variants may include, for example, single nucleotide variants (SNVs), indels, fusions, and/or copy number variation. Any known technique for variant calling may be used. In an embodiment, nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Any data analyzed, determined, and/or output by the sequence analysis pipeline 212 may be stored in the analysis datastore 218. Generally speaking, the processor 220 may implement (be programmed by) various components of the sequence analysis pipeline 212, such as the sequence quality control component 213, the pre-processor 214, the alignment component 215, the cluster component 216, the allele caller 217, the variant caller 219, and/or other components. Alternatively, it should be noted that these components of the sequence analysis pipeline 212 may include a hardware module. Although illustrated separately for convenience, one or more of the various components or instructions, such as the sequence quality control component 213, the pre-processor 214, the alignment component 215, the cluster component 216, the allele caller 217, and/or the variant caller 219 may be integrated with one another.

The computer system 210 may exchange data with a computer system 224 using a network 223. For example, the computer system 224 may retrieve data from the analytics datastore 218. The computer system 224 may be configured for determining/classifying alleles present at a locus.

Determining, based on the numbers of sequence reads that aligned to each known allele sequence, for the one or more loci, the known allele sequences present at the one or more loci may comprise determining one or more known allele sequences having a highest number of sequence reads aligned. Determining, based on the numbers of sequence read families that aligned to each known allele sequence, for the one or more loci, the known allele sequences present at the one or more loci may comprise determining one or more known allele sequences having a highest number of sequence read families aligned.

Generating the germline alignment of the plurality of pairs of sequence reads to a plurality of known allele sequences may comprise determining, based on the germline alignment, for a pair of sequence reads of the plurality of pairs of sequence reads, one or more known allele sequences to which each read of the pair of sequence reads aligns with no mismatch or indel.

Generating the decoy alignment of the plurality of pairs of sequence reads to a plurality of decoy allele sequences may comprise determining, based on the decoy alignment, for the pair of sequence reads of the plurality of pairs of sequence reads, one or more decoy allele sequences to which each read of the pair of sequence reads aligns with no mismatch or indel and discarding the pair of sequence reads.

Generating the decoy alignment of the plurality of pairs of sequence reads to a plurality of decoy allele sequences may comprise determining, based on the decoy alignment, for the pair of sequence reads of the plurality of pairs of sequence reads, one or more non-human decoy sequences to which each read of the pair of sequence reads aligns with no mismatch or indel and identifying the plurality of pairs of sequence reads as originating from a contaminated sample.

Generating the germline alignment of the plurality of pairs of sequence reads to a plurality of known allele sequences may comprise determining, based on the germline alignment, for a pair of sequence reads of the plurality of pairs of sequence reads, one or more known allele sequences to which each read of the pair of sequence reads aligns with at least one mismatch or indel and generating the germline alignment score.

Generating the decoy alignment of the plurality of pairs of sequence reads to a plurality of decoy allele sequences may comprise determining, based on the decoy alignment, for the pair of sequence reads of the plurality of pairs of sequence reads, one or more decoy allele sequences to which each read of the pair of sequence reads aligns with at least one mismatch or indel and generating the decoy alignment score.

Generating a germline alignment of the plurality of pairs of sequence reads to a plurality of known allele sequences may comprise determining a pair of sequence reads aligns to at least two allele sequences of the plurality of known allele sequences and selecting one known allele sequence of the at least two allele sequences.

Generating a decoy alignment of the plurality of pairs of sequence reads to a plurality of decoy allele sequences may comprise determining a pair of sequence reads align to at least two decoy allele sequences of the plurality of decoy allele sequences and selecting one decoy allele sequence of the at least two decoy allele sequences.

### IV. SYSTEMS AND COMPUTER READABLE MEDIA

The various processing operations and/or methods depicted in the Figures may be accomplished using some or all of the system components described in detail herein and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail herein) are provided as example and, as such, should not be viewed as limiting.

The present methods can be computer-implemented, such that any or all of the operations described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. Returning to FIG. 2, the processor 220 may include a single core or multi core processor, or a plurality of processors for parallel processing. The storage device 222 may include random-access memory, read-only memory, flash memory, a hard disk, and/or other type of storage. The computer system 210 may include a communication interface (e.g., network adapter) for communicating with one or more other systems, and peripheral devices, such as cache, other memory, data storage and/or electronic display adapters. The components of the computer system 210 may communicate with one another through an internal communication bus, such as a motherboard. The storage device 222 may be a data storage unit (or data repository) for storing data. The computer system 210 may be operatively coupled to a network 223 (“network”) with the aid of the communication interface. The network 223 may be the Internet, an internet and/or extranet, or an intranet and/or extranet that is in communication with the Internet. The network 223 in some cases is a telecommunication and/or data network. The network 223 may include a local area network. The network 23 may include one or more computer servers, which can enable distributed computing, such as cloud computing. The network 223, in some cases with the aid of the computer system 210, may implement a peer-to-peer network, which may enable devices coupled to the computer system 220 to behave as a client or a server. The computer system 210 may exchange data with a computer system 224 using the network 223. For example, the computer system 224 may retrieve data from the analytics datastore 218.

The processor 220 may execute a sequence of machine-readable instructions, which can be embodied in a program or software. The instructions may be stored in a memory location, such as the storage device 222. The instructions can be directed to the processor 220, which can subsequently program or otherwise configure the processor 220 to implement methods of the present disclosure. Examples of operations performed by the processor 220 may include fetch, decode, execute, and writeback.

The processor 220 may be part of a circuit, such as an integrated circuit. One or more other components of the system 200 may be included in the circuit. In some cases, the circuit may include an application specific integrated circuit (ASIC).

The storage device 222 may store files, such as drivers, libraries, and saved programs. The storage device 222 can store user data, e.g., user preferences and user programs. The computer system 210 in some cases may include one or more additional data storage units that are external to the computer system 210, such as located on a remote server that is in communication with the computer system 210 through an intranet or the Internet.

The computer system 210 can communicate with one or more remote computer systems through the network. For instance, the computer system 210 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 210 via the network.

Methods as described herein can be implemented by way of machine (e.g., computer processor) executable code stored on an electronic storage location of the computer system 210, such as, for example, on the storage device 222. The machine executable or machine readable code can be provided in the form of software (e.g., computer readable media). During use, the code can be executed by the processor 220. In some cases, the code can be retrieved from the storage device 222 and stored on the storage device 222 for ready access by the processor 220.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 210, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 210 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 220.

### V. EXAMPLES

### Example 1—CYP2D6 Genotyping Challenges

In some instances, allele calling in CYP2D6 can be aking to allele calling in highly homologous genes such as HLA or KIR. However, in some instances, genotyping CYP2D6 is complicated by several factors, such as unique tandem structure described and high homology to neighboring regions. First, CYP2D7 is almost identical to CYP2D6. Second, in some cases (such as *10.002+*36.004) the two alleles are often almost identical, and the majority of supporting reads are actually supporting both alleles. Third, whereas a filter can remove a read pair if it maps (perfectly) on more than one gene, in the case of CYP2D6, filter removal triggers false positives in the regular, conventional cases, due to the fact that the reads belonging to CYP2D7 randomly support odd CYP2D6 alleles. However, failure to remove the filter can lead to complete failure to account for the the few reads in the small region of *36 which is different from *10 (and which matches perfectly CYP2D7).

One can specifically look for these events, rather than trying to solve the general problem. For example, there are a limited number of known tandem arrangements and complex CNV structures. Additionally, the identification of the exact tandem arrangement or CNV structure is simply a means to an end: the clinically relevant aspect is the function of the gene (if normal, increased, or decreased). For example, calling *17 rather than *17+*17.001 would not impact the clinical function (in other words, one may decide not to try to identify this specific arrangement, since this would not change the clinical impact).

### Example 2—Design Organization

The process for detecting CYP2D6 alleles of complex arrangements involves a gene-based filter, unique reads pairs, and a ratio between unique read pairs.
(1) The gene-based filter is the name of the logic that removes a read pair if it maps perfectly on more than one gene (for example, both CYP2D6 and CYP2D7).
(2) Given an heterozygous gene, the unique read pairs for the two alleles are the two set of read pairs unique to each allele (this is relevant because it is often the case that a read pair supports both alleles).
(3) Given an heterozygous gene, the unique ratio is the ratio between the largest and smallest number of unique read pairs of the two alleles (hence, this is a real number>=1). This is defined to be zero if one of the two alleles has no unique read pairs.

### Example 3—List of Known Cases

Generate a list of known tandem rearrangements and copy number alterations. generate a list of special alleles, built from the list of known tandem rearrangements and copy number alterations. For example, if the known tandem rearrangement allele is CYP2D6*10.002+CYP2D6*36.004, then add CYP2D6*36.004 to the list of special alleles.

### Example 4—Run Kmerizer

Run the allele caller kmerizer, in particular, deploy the gene-based filter (this is the default behavior). In parallel, keep track of all special alleles, and remove (i.e., turn off) the gene-based filter for the supporting reads (this means that read pairs supporting multiple genes are allowed to support the special alleles). For example, to identify the hybrid *10.002+*36.004, one would need need to keep track of *36.004. Turn off the gene-based filter on the special alleles.

### Example 5—Match Alleles Against Known List

If one or both of the alleles called by kmerizer match one of the alleles in the list of known cases, check if one of the combinations is supported by reads, and that the unique ratio between the two is positive, and smaller than or equal to a user-defined threshold. More specifically, when kmerizer calls the germline events, check if one or more of the germline alleles could possibly fit one of the known arrangements. For example, if *10.002 is one of the alleles, check if *10.002+*36.004 is supported. If a combination is detected, then call it if and only if the unique ratio between the two alleles involved is both non-zero, and <=10.

### Example 6—Performance Evaluation

To evaluate the performance characteristics of the above, the Inventors sequenced several samples from Coriell's cell lines. In particular, two samples from cell line NA23090, with known CYP2D6 status *1, and *10.002+*36.004. The original algorithm identifies *1 and *10.002 as the “regular” alleles. Since *10.002 is one of the alleles in our list, the algorithm checks support for the special allele *36.004, and it checks that this allele is also supported. Then, the algorithm computes the unique ratio between *10.002 and *36.004 (˜4.5).⇒The algorithm calls *10.002+*36.004 as the second allele. As such, The algorithm correctly identified the two hybrid cases. The algorithm also called as tandem arrangements the two samples from cell line NA17248

Expected: *6, and *10. Called: *6, and *10.002+*36.004.

### Example 7—Clinical Functions

Correctly identify tandem arrangements and complex CNV structures in CYP2D6 is possible, although some cases may be hard to classify unambiguously.

In the same spirit as kmerizer (which relies on a list of known alleles to call genes), the logic would only match a sample's status against a list of known arrangements.

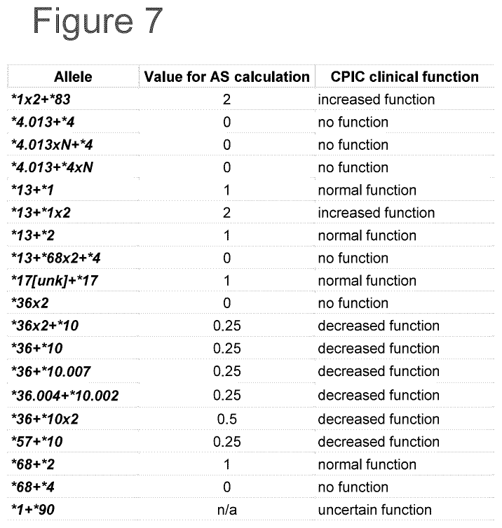

Even if in same cases it may be hard/impossible to classify exactly the allele status of a sample, it is likely possible to accurately predict the clinical function of the alleles in the sample. As shown in FIG. 7, alleles and ascertained clinical function can be degenerated. In such instances, the output from kmerizer is combined with the output from the CNV caller.

### Example 8

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, and determining, based on the numbers of sequence reads that aligned to each known allele sequence, for the one or more loci, the known allele sequences present at the one or more loci.

1. A computer implemented method comprising:
receiving a plurality of pairs of sequence reads from a sample of a subject;
aligning to a reference the plurality of pairs of sequence reads;
keeping all possible alignments of the pairs of sequence reads; and
calling one or more alleles.

2. The method of claim 1, further comprising:
obtaining the sample from the subject; and
sequencing the sample to obtain the plurality of pairs of sequence reads.

3. The method of claim 1, wherein the reference comprises all known alleles of the Cytochrome P450 Family 2 Subfamily D (CYP2D) family of genes.

4. The method of claim 3, wherein the CYP2D family of genes comprises at least CYP2D6, CYP2D7, and CYP2D78P.

5. The method of claim 3, wherein the reference comprises all known alleles of the cytochrome P450 enzymes.

6. The method of claim 5, wherein the cytochrome P450 enzymes comprise at least: CYP2D6, CYP2D7, CYP2D78P, CYP3A4, CYP3A5, CYP1A1, CYP1A2, CYP2C9, CYP2C19, CYP2E1, CYP2F1, CYP2J2, CYP1B1, CYP2A6, CYP2B6, CYP2C8, CYP2C18, CYP2C29, CYP2D8P, CYP2D78, CYP2R1, CYP2S1, CYP2U1, CYP2W1, CYP2Y1, CYP2Y11, CYP2Y12, CYP2Y13, CYP2Y8, CYP2Y9, CYP4A11, CYP4A22, CYP4B1, CYP4F11, CYP4F12, CYP4F22, CYP4V2, CYP4X1, CYP4Z1, CYP4F2, CYP4F3, CYP4F8, CYP4F12, CYP4F22, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, CYP4F2, CYP4F3, CYP4F8, CYP4F12, CYP4F22, CYP4V2, CYP4X1, CYP4Z1, and CYP4F2.

7. The method of claim 1, wherein the reference is built by concatenating the sequences of all the known alleles of the CYP2D family of genes.

8. The method of claim 1, further comprising:
generating a lists of all possible alignments of the plurality of pairs of sequence reads against the reference.

9. The method of claim 8, further comprising sorting all possible alignments into weak and strong alignments.

10. The method of claim 9, wherein strong alignments comprise read pairs aligning to a single gene, and weak alignments comprise read pairs aligning to different genes.

11. The method of claim 1, wherein the calling comprises (1) determining a combination from the reference that best matches the alleles, and (2) calling heterozygous alleles when a unique ratio between the reads supporting a first allele and the reads supporting the second allele does not exceed a set threshold.

12. The method of claim 11, wherein the unique ratio comprises the ratio between the largest and smallest number of unique read pairs of the two alleles.

13. The method of claim 11, wherein the unique ratio is both non-zero and less than or equal to 10.

14. The method of claims 12, wherein the unique read pairs for the two alleles comprise the set of read pairs unique to each allele.

15. The method of claim 1, wherein the reference comprises at least a portion of chromosomal position 22q13.2.

16. The method of claim 1, wherein the sample is cell free DNA (cfDNA).

17-23. (canceled)

24. A method of genotyping a nucleic acid sequence, comprising:
receiving a plurality of pairs of sequence reads from a sample of a subject;
aligning to a reference the plurality of pairs of sequence reads;
keeping all possible alignments of the pairs of sequence reads;
categorizing alignments into strong and weak alignments; and
calling one or more alleles, wherein the nucleic acid sequence comprises one or more confounding features.

25. The method of claim 24, wherein the confounding features comprise at least one tandem arrangement, copy number amplification (CNA) and/or complex CNV structures.

26. The method of claim 25, wherein the tandem arrangement comprises a duplication of CYP2D6.

27. The method of claim 25, wherein the duplication of CYP2D6 comprises alleles CYP2D6*36 and CYP2D6*10 in tandem arrangement.

28. A method, comprising:
determining a plurality of sequence read pairs of a target region of a chromosome of a subject, wherein the target region of the chromosome comprises one or more loci, aligning the plurality of sequence read pairs to a plurality of known allele sequences,
determining, based on the alignment, for each known allele sequence of the plurality of known allele sequences, strong read pairs aligning to a single gene, and weak pairs to aligning different genes,
determining a combination from the reference that best matches the alleles strong read pairs and weak read pairs,
calling heterozygous alleles when a unique ratio between the reads supporting one allele and the reads supporting the second allele does not exceed a set threshold.

29-32. (canceled)
